(19) United States
(12) Patent Application Publication
(10) Pub. No.: US 2023/0346243 A1
(43) Pub. Date: Nov. 2, 2023

(54) Magnet Arrangement for a Magnetic Resonance Apparatus

(71) Applicant: Siemens Healthcare GmbH, Erlangen (DE)

(72) Inventors: Swen Campagna, Engelthal (DE); Andreas Krug, Fürth (DE)

(73) Assignee: Siemens Healthcare GmbH, Erlangen (DE)

(21) Appl. No.: 18/137,568

(22) Filed: Apr. 21, 2023

(30) Foreign Application Priority Data
Apr. 27, 2022 (EP) 22170324.2

(51) Int. Cl. A61B 5/055; G01R 33/385

(52) U.S. Cl. CPC A61B 5/055; G01R 33/385

(57) ABSTRACT

A magnet arrangement for a magnetic resonance apparatus for capturing magnetic resonance data from an object may include gradient coils and gradient amplifiers. The gradient amplifiers may be configured to variably set a current flow in the gradient coils. Each gradient coil of the gradient arrangement may be electrically connected to a gradient amplifier of the plurality of gradient amplifiers. The magnet arrangement may provide, by the gradient amplifiers, in an alternating manner, a substantially homogeneous magnetic field and a magnetic gradient field.

### CROSS REFERENCE TO RELATED APPLICATIONS

This patent application claims priority to European Patent Application No. 22170324.2, filed Apr. 27, 2022, which is incorporated herein by reference in its entirety.

### BACKGROUND

### Field

The disclosure relates to a magnet arrangement for a magnetic resonance apparatus for capturing magnetic resonance data from an object. The disclosure further relates to a magnetic resonance apparatus for capturing magnetic resonance data from an object, comprising a magnet arrangement and a controller.

### Related Art

Magnetic resonance tomography is a known imaging method with which magnetic resonance images of an interior of the examination object can be generated. When carrying out a magnetic resonance imaging, the examination object is typically positioned in a strong, static and homogeneous main magnetic field (BO magnetic field) of a magnetic resonance apparatus. The main magnetic field can have magnetic field strengths from 0.2 tesla to 7 tesla, so that nuclear spins in the examination object become oriented along the main magnetic field. In order to cause so-called nuclear spin resonances, high frequency signals, known as excitation pulses (B1 magnetic field) are radiated into the examination object. Each excitation pulse causes a deviation of a magnetization of particular nuclear spins in the examination object from the main magnetic field by an amount that is known as the flip angle. An excitation pulse can be an alternating magnetic field with a frequency that corresponds to the Larmor frequency at the respective static magnetic field strength. The excited nuclear spins can have a rotating and decaying magnetization (nuclear spin resonance) which can be captured by means of special antennae as a magnetic resonance signal. For spatial encoding of the nuclear spin resonances of the examination object, magnetic gradient fields can be overlaid on the main magnetic field.

The magnetic resonance signals received are typically digitized and stored as complex values (magnetic resonance data) in a k-space matrix. This k-space matrix can be used as the basis for a reconstruction of magnetic resonance images and a determination of spectroscopic data.

The reconstruction of a magnetic resonance image typically takes place by means of a multi-dimensional Fourier transform of the k-space matrix.

Magnet arrangements in magnetic resonance apparatuses typically consist of main magnets for generating a static magnetic field and gradient coils for generating magnetic fields rising in a linear manner (magnetic gradient fields). Although the gradient coils are often designed as resistive coils, the main magnets can have different magnet types, for example, superconducting magnets, permanent magnets, electromagnets and suchlike.

It is desirable to provide smaller and/or dedicated magnetic resonance apparatuses with low magnetic field strengths (<1 tesla) which by reason of lower costs and/or more compact dimensions, can be used in smaller practices and clinical establishments, but also as dedicated systems for non-radiologists (e.g. dentistry practices, orthopedics, ophthalmic clinics and suchlike). However, the operation of magnetic resonance apparatuses, in particular those with superconducting main magnets requires an extensive technical infrastructure which has a significant space requirement. In addition, magnetic leakage fields from the main magnet necessitate significant restrictions on other uses in the direct vicinity of the magnetic resonance apparatus. In conventional magnetic resonance apparatuses, leakage fields are also active outside of the actual scan operation. Particularly in the case of superconducting main magnets, the static magnetic field must be maintained constantly, which requires continuous cooling and/or constant operation of suitable cooling devices.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 shows an example magnetic resonance apparatus.

FIG. 2 shows a schematic representation of current flows in an example gradient system.

FIG. 3 shows a magnet arrangement according to an exemplary embodiment of the disclosure.

FIG. 4 shows a magnet arrangement according to an exemplary embodiment of the disclosure.

FIG. 5 shows a magnet arrangement according to an exemplary embodiment of the disclosure.

FIG. 6 shows a schematic representation of current flows in gradient arrangements of a magnet arrangement according to an exemplary embodiment of the disclosure.

FIG. 7 shows a magnet arrangement according to an exemplary embodiment of the disclosure.

The exemplary embodiments of the present disclosure will be described with reference to the accompanying drawings. Elements, features and components that are identical, functionally identical and have the same effect are — insofar as is not stated otherwise — respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

The magnetic resonance apparatus may be operated for only a certain time per day, and the main magnet and the necessary cooling devices may be active only during this time. In this way, energy costs can be saved in the operation of the magnetic resonance apparatus.

An object of the disclosure is to provide a magnetic resonance apparatus which has a reduced energy demand during standby and/or idle periods.

The magnet arrangement according to the disclosure for a magnetic resonance apparatus for capturing magnetic resonance data from an object comprises at least one gradient arrangement with a plurality of gradient coils and a plurality of gradient amplifiers.

The magnet arrangement can be understood as a magnetic field-generating unit (magnetic field generator) of the magnetic resonance apparatus. In an exemplary embodiment, the magnet arrangement is designed to provide a magnetic field in a patient receiving region of the magnetic resonance apparatus. It is conceivable, in particular, that the magnet arrangement is designed to generate magnetic fields with varying magnetic field strength and/or magnetic field orientation in the patient receiving region. However, the magnet arrangement is also designed to generate a homogeneous magnetic field in the patient receiving region.

The gradient arrangement can be a part of a gradient system of the magnet arrangement. The gradient system can be designed to generate magnetic gradient fields in the patient receiving region. In particular, the gradient system can be designed to provide magnetic gradient fields which are oriented along different spatial directions. In an exemplary embodiment, the gradient system is designed to provide two or three magnetic gradient fields which are oriented orthogonally to one another.

The magnet arrangement can further comprise a high frequency system. The high frequency system can be designed to emit high frequency signals into an imaging region and/or to receive high frequency signals (magnetic resonance signals) from the imaging region.

The gradient system can comprise one or a plurality of gradient arrangements. A gradient arrangement can be understood to be a group or an arrangement of a plurality of gradient coils which are designed, by way of a cooperation, to provide, in an alternating manner, a homogenous magnetic field and a magnetic gradient field with a predetermined orientation in the imaging region.

The plurality of gradient amplifiers is designed to set a current flow in the plurality of gradient coils in a variable manner, wherein each gradient coil of the at least one gradient arrangement is electrically connected to a gradient amplifier of the at least one gradient arrangement.

A gradient amplifier is designed to generate a current flow in a gradient coil. The current flow in the gradient coil can therein correspond to a predetermined and/or specified electrical signal which is provided to the gradient amplifier. In an exemplary embodiment, each gradient amplifier of the at least one gradient arrangement is electrically connected to a controller of the magnetic resonance apparatus. It is conceivable that one or more gradient amplifiers of the at least one gradient arrangement are designed, dependent upon a signal from the controller, to provide a current flow in one or more gradient coils of the at least one gradient arrangement. The current flows in the one or more gradient coils can be provided simultaneously, or independently of one another.

In an exemplary embodiment, the at least one gradient arrangement has a first gradient coil and a second gradient coil. The first gradient coil can therein be electrically connected to a first gradient amplifier, while the second gradient coil is electrically connected to a second gradient amplifier. In an exemplary embodiment, the first gradient amplifier and the second gradient amplifier are different gradient amplifiers. The first gradient amplifier and the second gradient amplifier can be electrically unconnected. In an exemplary embodiment, each gradient coil of the at least one gradient arrangement is electrically connected to exactly one gradient amplifier of the at least one gradient arrangement.

However, it is conceivable that the at least one gradient arrangement has a plurality of gradient coils, for example, four, six, eight, ten or more gradient coils. In this case, a gradient amplifier can also be electrically connected to a plurality of gradient coils in order to generate a current flow in the plurality of gradient coils.

The magnet arrangement according to the disclosure is designed to provide, by means of the plurality of gradient amplifiers, in an alternating manner, a substantially homogeneous magnetic field and a magnetic gradient field. For example, the plurality of gradient amplifiers of the at least one gradient arrangement can be designed to adapt a current flow in the plurality of gradient coils in order, rather than the homogeneous magnetic field, to generate the magnetic gradient field.

However, it is equally conceivable that the magnet arrangement according to the disclosure comprises at least one further gradient arrangement, for example, a second gradient arrangement and/or a third gradient arrangement. In this case, a first gradient arrangement can be designed to provide the substantially homogeneous magnetic field, while a further gradient arrangement is designed to provide the magnetic gradient field. The provision of the magnetic gradient field can thus also comprise an overlaying of the substantially homogeneous magnetic field with the magnetic gradient field.

A magnetic gradient field can be characterized by a linear or non-linear magnetic field strength progression in at least one spatial direction. The magnetic gradient field can further be characterized by an overlaying of the substantially homogeneous magnetic field with the magnetic gradient field. It is further conceivable that the magnetic gradient field replaces the substantially homogeneous magnetic field.

The magnet arrangement according to the disclosure may be designed to provide the substantially homogeneous magnetic field and/or the magnetic gradient field as required. The substantially homogeneous magnetic field and the magnetic gradient field can be provided, for example, dependent upon a magnetic resonance examination, an imaging sequence, an imaging protocol and/or an imaging parameter in an alternating manner.

In an exemplary embodiment, a magnetic field strength of the substantially homogeneous magnetic field may amount to a few hundred millitesla. For example, the magnetic field strength of the substantially homogeneous magnetic field is between 0.1 T and 0.5 T or between 0.1 T and 0.3 T. A gradient field strength of the magnetic gradient field is, for example, in a region from a few tens of mT/m, in particular between 10 mT/m and 60 mT/m or preferably between 10 mT/m and 30 mT/m.

In an exemplary embodiment, the magnet arrangement and/or a magnetic resonance apparatus that comprises the magnet arrangement has a controller. The controller can be designed to actuate the at least one gradient arrangement and/or further gradient arrangements, the substantially homogeneous magnetic field or the magnetic gradient field.

The magnet arrangement according to the disclosure can replace, in particular, a conventional main magnet for generating a static, homogeneous magnetic field (B0). This can mean that the magnet arrangement according to the disclosure is able to provide a homogeneous magnetic field without a conventional or separate main magnet. The homogeneous magnetic field provided by the magnet arrangement according to the disclosure can advantageously be taken out of operation temporarily or for relatively long periods without the need for a complex ramp-down or ramp-up, as is known with superconducting magnets.

By means of the magnet arrangement according to the disclosure, operating times of the magnetic resonance apparatus can advantageously be adapted to an actual need for magnetic resonance examinations. In particular, standby or readiness periods in which conventional magnetic resonance apparatuses have a relatively high energy demand can be avoided.

Furthermore, effort is advantageously spared in the production and/or installation of a magnetic resonance apparatus by means of the use of the magnet arrangement according to the disclosure, since conventional main magnets with corresponding cooling devices can be dispensed with.

By dispensing with a conventional main magnet, technical equipment for screening the main magnet can also be omitted, so that the costs and dimensions of the magnetic resonance apparatus can advantageously be reduced.

Furthermore, a use of an examination space in which the magnetic resonance apparatus is accommodated can be improved and/or simplified in an advantageous manner by avoiding a conventional main magnet with a continuously active magnetic field.

In an exemplary embodiment of the magnet arrangement according to the disclosure, the at least one gradient arrangement comprises a first gradient coil and a second gradient coil.

In an exemplary embodiment, the at least one gradient arrangement has exactly two gradient coils. However, the at least one gradient arrangement can also comprise more than two gradient coils, in particular, three or four gradient coils.

The first gradient coil and the second gradient coil are substantially circular and planar and are arranged in planes oriented parallel and arranged separated from one another, such that a projection of a first area enclosed by the first gradient coil along a normal vector of the first area and a second area enclosed by the second gradient coil have a non-empty intersection.

The gradient coils of the at least one gradient arrangement can be designed, in particular, as parts of a Helmholtz or Maxwell arrangement. In the case of a Maxwell arrangement, the at least one gradient arrangement can have, in particular, a third gradient coil which is may be arranged between the first gradient coil and the second gradient coil.

In an exemplary embodiment, the magnet arrangement has a hollow cylindrical form. The first gradient coil and the second gradient coil can therein be arranged such that mid-points of the area enclosed by the first gradient coil and the second gradient coil are arranged on a cylindrical axis of the hollow cylindrical magnet arrangement. The first gradient coil and the second gradient coil may be positioned at opposite ends of the hollow cylindrical magnet arrangement. It is conceivable that the first gradient coil and the second gradient coil enclose a patient receiving region, which is formed by the cylindrical magnet arrangement, along a circumferential direction.

However, the shape of the magnet arrangement can also deviate from a hollow cylinder. In this case, the first gradient coil and the second gradient coil may be arranged on two opposite sides of the patient receiving region. The magnetic resonance apparatus can herein be designed, in particular, as a “C” scanner and/or an “open” scanner.

The first gradient coil is electrically connected to a first gradient amplifier, and the second gradient coil is electrically connected to a second gradient amplifier. In an exemplary embodiment, the first gradient coil is electrically separated from the second gradient coil.

The first gradient amplifier can be designed to set a current flow in the first gradient coil. In an exemplary embodiment, the first gradient amplifier is designed to set and/or adjust a current strength and/or a voltage, for example, dependent upon a control signal from a controller, processor, computer, or the like. The second gradient amplifier can likewise be designed to set a current strength and/or a voltage of a current flow through the second gradient coil. The current flows through the first gradient coil and the second gradient coil can have the same or different sign. In an exemplary embodiment, the first gradient coil and the second gradient coil are designed to provide, by means of the first gradient amplifier and the second gradient amplifier, in an alternating manner, a homogeneous magnetic field (BO field) and a magnetic gradient field. The magnetic gradient field can herein be oriented, in particular, along the cylinder axis (e.g. a z-direction) of the hollow cylindrical magnet arrangement.

By way of the provision of the first gradient coil with the first gradient amplifier and of the second gradient coil with the second gradient amplifier, a current flow through the first gradient coil can advantageously be set independently of the current flow through the second gradient coil. In this way, a homogeneous magnetic field or a magnetic gradient field can be set in an imaging region dependent upon requirements of an imaging sequence. The imaging region can substantially coincide with a patient receiving region of a magnetic resonance apparatus for human or animal patients. The magnetic resonance apparatus can equally, however, be adapted for the examination of any objects, for example, archeological finds or foods and can have a correspondingly dimensioned imaging region.

In a further embodiment, the magnet arrangement according to the disclosure further comprises a second gradient arrangement with a plurality of second gradient coils and a plurality of second gradient amplifiers, wherein at least two gradient coils of the plurality of second gradient coils are each electrically connected to a gradient amplifier of the plurality of second gradient amplifiers.

In an exemplary embodiment, a first gradient amplifier of the second gradient arrangement is electrically connected to a first gradient coil of the second gradient arrangement, whereas a second gradient amplifier of the second gradient arrangement is electrically connected to a second gradient coil of the second gradient arrangement. In an exemplary embodiment, the first gradient coil and the second gradient coil of the second gradient arrangement are separated from one another electrically.

The magnet arrangement is designed to provide, by means of the plurality of second gradient amplifiers, in an alternating manner, a substantially homogeneous magnetic field and a second magnetic gradient field, wherein an orientation of the second magnetic gradient field differs from an orientation of the magnetic gradient field.

In an exemplary embodiment, the second magnetic gradient field is oriented substantially orthogonally to the magnetic gradient field.

As described above, the first gradient amplifier and the second gradient amplifier of the second gradient arrangement can be designed to set symmetrical current flows with the same sign in the first gradient coil and the second gradient coil of the second gradient arrangement. The magnetic field thus generated by the second gradient arrangement can be, in particular, a homogeneous magnetic field. In addition, the first gradient amplifier and the second gradient amplifier of the second gradient arrangement are designed to set different current flows and/or current flows of different sign in the first gradient coil and the second gradient coil of the second gradient arrangement. The magnetic field thus generated can represent, in particular, a second magnetic gradient field which is oriented substantially orthogonally to the magnetic gradient field.

In a further embodiment, the magnet arrangement according to the disclosure further comprises a third gradient arrangement with a plurality of third gradient coils and a plurality of third gradient amplifiers. At least two gradient coils of the plurality of third gradient coils are each electrically connected to a gradient amplifier of the plurality of third gradient amplifiers.

According to an embodiment described above, a first gradient amplifier of the third gradient arrangement can be electrically connected to a first gradient coil of the third gradient arrangement, whereas a second gradient amplifier of the third gradient arrangement is electrically connected to a second gradient coil of the third gradient arrangement. The first gradient coil and the second gradient coil of the third gradient arrangement can be separated from one another, in particular, electrically.

The magnet arrangement is designed to provide, by means of the plurality of third gradient amplifiers, in an alternating manner, a substantially homogeneous magnetic field and a third magnetic gradient field, wherein an orientation of the third magnetic gradient field differs from an orientation of the magnetic gradient field and/or of the second magnetic field.

In an exemplary embodiment, the third magnetic gradient field is oriented substantially orthogonally to the magnetic gradient field and/or to the second magnetic gradient field.

The first gradient amplifier and the second gradient amplifier of the third gradient arrangement can be designed to set symmetrical current flows with the same sign in the first gradient coil and the second gradient coil of the third gradient arrangement. The magnetic field thus generated by the third gradient arrangement can be, in particular, a homogeneous magnetic field. It is also conceivable that the first gradient amplifier and the second gradient amplifier of the third gradient arrangement are designed to set asymmetrical current flows in the first gradient coil and the second gradient coil of the third gradient arrangement, such as for example, different current flows and/or current flows of different sign. The magnetic field thus generated can represent, in particular, a third magnetic gradient field which is oriented orthogonally to the magnetic gradient field and the second magnetic gradient field.

By way of the provision of the second gradient arrangement and/or the third gradient arrangement, a magnetic field strength of a homogeneous magnetic field provided by the at least one gradient arrangement can advantageously be increased by overlaying with a homogeneous magnetic field provided by the second gradient arrangement and/or the third gradient arrangement. Thereby, technical requirements of the at least one gradient arrangement can advantageously be reduced. Furthermore, by means of the second gradient arrangement and/or the third gradient arrangement, a second magnetic gradient field and/or a third magnetic gradient field, which can advantageously be used for a spatial allocation of received magnetic resonance signals, can be provided, at least briefly.

In an exemplary embodiment of the magnet arrangement according to the disclosure, the plurality of second gradient coils and/or the plurality of third gradient coils are designed as saddle coils.

Saddle coils can have the form of a cylindrical shell, in particular a hemicylindrical shell. Saddle coils can comprise electrically conductive signal conductors which can be arranged in a “fingerprint” pattern or a comparable winding pattern. The saddle coils can also be designed as so-called Golay coils. In an exemplary embodiment, the second plurality of gradient coils and the third plurality of gradient coils are arranged in the magnet arrangement such that the saddle coils enclose a cylindrical volume which substantially coincides with the imaging region of the magnet arrangement.

In an alternative embodiment of the magnet arrangement according to the disclosure, the plurality of second gradient coils and/or the plurality of third gradient coils are designed as segment coils. A segment coil can be designed such that a current in a field-generating conductor of the segment coil flows back on a cylindrical surface with a larger radius. This can mean that the segment coil has return conductors (secondary conductors) which are arranged offset substantially parallel to service conductors (primary conductors) in a side of the service conductors facing away from the imaging region.

By way of the provision of a segment coil, the return conductors can be arranged, in an advantageous manner, axially displaced on the outer cylindrical surface in order to improve the homogeneity of a generated magnetic field.

According to a further embodiment, the magnet arrangement according to the disclosure has a substantially hollow cylindrical shape.

The magnet arrangement can, in particular, have the form of a tube and/or a hollow cylinder. Advantageously, the hollow cylindrical magnet arrangement may be designed such that it encloses an imaging volume within the imaging region and/or the patient receiving region along a circumferential direction.

At least one gradient coil of the second gradient arrangement comprises a first coil portion and a second coil portion. The first coil portion and the second coil portion are arranged disjoint from one another along an axial direction of the hollow cylindrical magnet arrangement, following one another.

The first coil portion can, for example, represent a separate winding portion or a separate subportion of the at least one gradient coil. This can mean that the first coil portion and the second coil portion are spaced from one another. In an exemplary embodiment, a winding of the first coil portion may be spatially separate from a winding of the second coil portion of the at least one gradient coil. The at least one gradient coil can further consist of a plurality of disjoint coil portions, for example, two, three, four or more coil portions.

In an exemplary embodiment, the first coil portion and the second coil portion are arranged on a common cylinder half of the hollow cylindrical magnet arrangement. It is conceivable, in particular, that the first coil portion and the second coil portion are arranged on two substantially mirror-symmetric cylinder portions of the hollow cylindrical magnet arrangement or the hollow cylindrical magnet arrangement is subdivided into two substantially mirror symmetrical cylinder portions.

The first coil portion and the second coil portion are jointly electrically connected to exactly one gradient amplifier of the second gradient arrangement.

In one embodiment, a gradient coil of the third gradient arrangement can also comprise a first coil portion and a second coil portion, wherein the first coil portion and the second coil portion are arranged disjoint from one another along an axial direction of the hollow cylindrical magnet arrangement, following one another. In an exemplary embodiment, the first coil portion and the second coil portion of the gradient coil of the third gradient arrangement are electrically connected to exactly one gradient amplifier of the third gradient arrangement. The first coil portion and the second coil portion of the gradient coil of the third gradient arrangement can be arranged similarly to the coil portions of the second gradient arrangement. In an exemplary embodiment, the coil portions of the third gradient arrangement are arranged offset by an angle of approximately 90° relative to the coil portions of the second gradient arrangement.

Furthermore, a second gradient coil of the second gradient arrangement can also have a first coil portion and a second coil portion according to an embodiment described above. It is conceivable that the first coil portion of the at least one gradient coil and the first coil portion of the further gradient coil are opposingly positioned along the axial direction of the hollow cylindrical magnet arrangement. In the same way, the second coil portion of the at least one gradient coil and the second coil portion of the further gradient coil are arranged opposingly positioned along the axial direction of the hollow cylindrical magnet arrangement.

In an alternative embodiment, the magnet arrangement has a form deviating from a hollow cylinder. For example, the magnet arrangement can also be designed C-shaped, U-shaped or V-shaped. It is further conceivable that the patient receiving region formed by the magnet arrangement is adapted to a shape of a body region of a patient. For example, the magnet arrangement can be adapted to a head, a hip, a shoulder, a chest, a knee, an arm and/or a leg of a patient.

The first coil portion and/or the second coil portion of the second gradient arrangement and/or of the third gradient arrangement can further be subdivided into two, three, four or more subportions. In an exemplary embodiment, the plurality of subportions of the first coil portion are arranged following one another along the axial direction of the hollow cylindrical magnet arrangement. In the same way, the plurality of subportions of the second coil portion can also be arranged following one another along the axial direction of the hollow cylindrical magnet arrangement.

The plurality of subportions of the first coil portion is electrically connected to a first gradient amplifier.

In the same way, the plurality of subportions of the second coil portion can be electrically connected to a second gradient amplifier. The first gradient amplifier and the second gradient amplifier therein differ from one another.

By way of the provision of coil portions with a plurality of subportions, a production complexity of the magnet arrangement can advantageously be reduced. Furthermore, a gradient arrangement with a plurality of coil portions, but also a magnetic field generated by the gradient arrangement, can be better adapted to a geometry of a dedicated magnetic resonance apparatus for a magnetic resonance examination of specific objects.

In a further embodiment of the magnet arrangement according to the disclosure, at least one gradient coil of the at least one gradient arrangement, the second gradient arrangement and/or the third gradient arrangement has a secondary coil. The secondary coil is arranged substantially parallel to the at least one gradient coil.

A three-dimensional form, a dimension, a geometrical form, but also a winding pattern of the secondary coil can substantially match a three-dimensional form, a dimension, a geometrical form and/or a winding pattern of the at least one gradient coil.

The secondary coil borders the at least one gradient coil in a direction facing away from the imaging region of the magnet arrangement. This can mean that the secondary coil encloses or encompasses the at least one gradient coil on a side facing away from the imaging volume. In an exemplary embodiment, the secondary coil and the at least one gradient coil are therein spaced from one another. A spacing between the secondary coil and the at least one gradient coil can amount, for example, to multiple millimeters or multiple centimeters.

If the at least one gradient coil consists of a plurality of coil portions and/or a plurality of subportions, then the secondary coil can also have a corresponding number of coil portions and/or subportions.

In an exemplary embodiment, the secondary coil and the at least one gradient coil are jointly electrically connected to a gradient amplifier. This can mean that a current flow through the secondary coil and the at least one gradient coil is able to be set jointly by means of an output signal from the gradient amplifier.

By way of the provision of a secondary coil, a magnetic field strength of a homogeneous magnetic field that is created or a magnetic gradient field of the magnet arrangement according to the disclosure can advantageously be increased. For example, in comparison with conventional gradient coils in which the secondary coils screen a generated magnetic gradient field outside the gradient coil (typically in the direction of the main magnet), a secondary coil of the magnet arrangement according to the disclosure can advantageously serve to amplify a magnetic field strength in the imaging region. Electromagnetic screening of the gradient arrangement(s) can therein advantageously be dispensed with.

In an exemplary embodiment of the magnet arrangement according to the disclosure, the at least one gradient arrangement, the second gradient arrangement and/or the third gradient arrangement is electromagnetically unscreened.

In conventional magnetic resonance apparatuses, an electromagnetic screening protects, inter alia, the main magnet, but also a surrounding area of the magnetic resonance apparatus, against electromagnetic fields. In particular, in conventional magnetic resonance apparatuses, a screening system is arranged between the gradient system and the (superconducting) main magnet. Thereby, effects of the gradient coils on the main magnet, for example eddy currents, can be reduced or prevented.

The electromagnetic screening of conventional magnetic resonance apparatuses typically has electrically conductive structures such as, for example, highly conductive metals or superconducting materials. In an exemplary embodiment, the use of such electrically conductive structures may be dispensed with in the magnet arrangement according to the disclosure, since no main magnet is present. In addition, the magnet arrangement according to the disclosure can be transferred into a readiness or standby operation, advantageously in a magnetic field-free state. Thus, a use of the examination space of the magnetic resonance apparatus is advantageously enabled even without using an electromagnetic screening.

According to an exemplary embodiment, the at least one gradient arrangement, the second gradient arrangement and/or the third gradient arrangement of the magnet arrangement according to the disclosure replace a main magnet for generating a static, homogeneous magnetic field.

A main magnet of conventional magnetic resonance apparatuses is characterized, in particular, by the generation of a static magnetic field. The static magnetic field is typically present constantly, e.g. also when the magnetic resonance apparatus is in a standby mode. An orderly shut-down of the main magnet is therein associated with a large amount of effort.

Equally, re-starting of the main magnet is associated with a high cost in time since the main magnet heats up when taken out of use and must first be cooled down to a superconducting temperature level.

However, the magnetic field generated by the at least one gradient arrangement, the second gradient arrangement and/or the third gradient arrangement of the magnet arrangement according to the disclosure is taken or switched out of operation in just a short time.

A replacement of the main magnet by the at least one gradient arrangement, the second gradient arrangement and/or the third gradient arrangement can mean, in particular, that the main magnet is dispensed with. The magnet arrangement according to the disclosure and also the magnetic resonance apparatus according to the disclosure can be characterized in that they have no main magnet or dispense with a main magnet. In an exemplary embodiment, in the magnet arrangement according to the disclosure and/or the magnetic resonance apparatus according to the disclosure, the provision of a static, homogeneous magnetic field takes place by means of the at least one gradient arrangement, the second gradient arrangement and/or the third gradient arrangement.

By dispensing with the electromagnetic screening and also the main magnet, a weight, a production effort and also a material usage for the magnet arrangement according to the disclosure can advantageously be reduced as compared with a conventional magnet arrangement.

The magnetic resonance apparatus according to the disclosure for capturing magnetic resonance data from an object comprises a magnet arrangement according to an embodiment described above and a controller which is designed to actuate a plurality of gradient amplifiers to set a current flow in a plurality of gradient coils variably in order to provide, in an alternating manner, a substantially homogeneous magnetic field and a magnetic gradient field in an imaging region of the magnet arrangement.

An object can be, for example, a human or animal body or any desired object. In an exemplary embodiment, the object is a human patient.

The controller of the magnetic resonance apparatus has a signal connection to the plurality of gradient amplifiers. In an exemplary embodiment, the controller is designed to actuate individual gradient amplifiers of the plurality of gradient amplifiers individually by means of the signal connection in order to set or specify the current flow in gradient coils electrically connected to the gradient amplifiers.

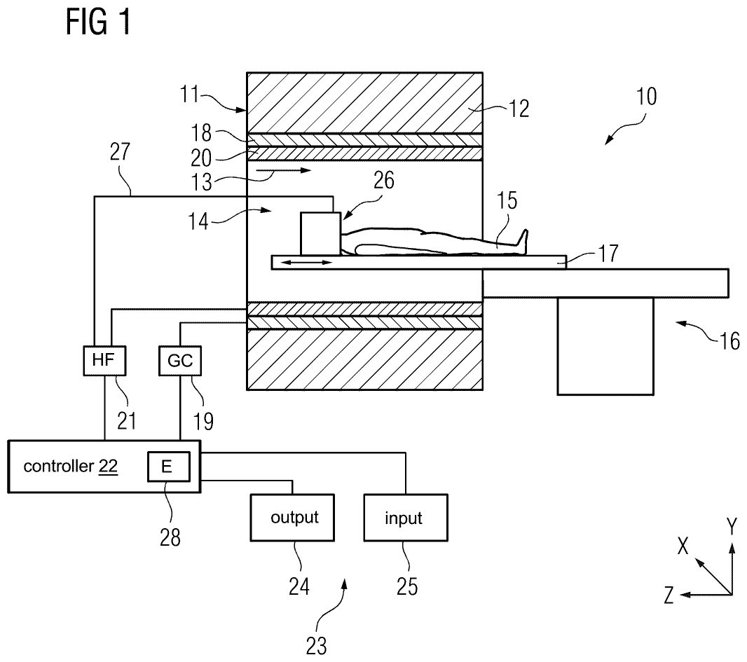

The controller 22 (FIG. 1) can be integrated into the magnetic resonance apparatus or can be configured as a standalone component. In an exemplary embodiment, the controller 22 has a signal connection to a computer of the magnetic resonance apparatus. It is conceivable that the controller is designed to adapt the current flow through the at least one gradient arrangement to a desired imaging sequence or a magnetic resonance data capture protocol independently or dependent upon a signal of the computer. The desired imaging sequence or the magnetic resonance data capture protocol can herein be specified by a user of the magnetic resonance apparatus, for example to carry out a magnetic resonance examination of the object.

The magnetic resonance examination according to the disclosure shares the advantages of the magnet arrangement according to the disclosure. By way of the provision of the controller, the current flows through the gradient arrangement of the magnet arrangement can advantageously be adapted to a requirement of a magnetic resonance examination. For example, a magnetic field strength of a generated homogeneous magnetic field can be adapted dependent upon the object and/or the magnetic resonance examination to be carried out. In this way, an energy requirement of the magnetic resonance apparatus, but also a quality of captured magnetic resonance data can be improved or optimized dependent upon various requirements.

In an embodiment of the magnetic resonance apparatus according to the disclosure, the controller is designed to convert symmetrical current flows through a plurality of gradient coils of a first gradient arrangement by means of a plurality of gradient amplifiers of the first gradient arrangement into asymmetrical current flows in order to provide a changeover from a (pure) homogeneous magnetic field to a magnetic gradient field.

In particular, the current flows through the plurality of gradient coils each have portions or gradient terms which are substantially identical during the provision of a homogeneous magnetic field, but differ during the provision of a magnetic gradient field. A gradient term can therein be characterized by a portion of the current flow through a gradient coil or can constitute a portion of a current flow through a gradient coil. In the presence of a magnetic gradient field, a first gradient term of a first gradient coil can have, for example, a negative sign, whereas a second gradient term of a second gradient coil has a positive sign. In this case, a difference between the current flows through the first gradient coil and the second gradient coil can be characterized by a sum of the amounts of the gradient terms of the first gradient coil and the second gradient coil. By contrast thereto, a difference between the current flows through the first gradient coil and the second gradient coil during provision of a homogeneous magnetic field can substantially correspond to the value zero.

By providing a controller which is designed to change or invert small portions of current flows through a plurality of gradient coils, a switching of large currents on transition from a homogeneous magnetic field to a magnetic gradient field can advantageously be avoided.

According to a further embodiment, the controller of the magnetic resonance apparatus according to the disclosure is designed to actuate at least one gradient amplifier of a first gradient arrangement to set a current flow through the first gradient arrangement in order to provide a magnetic gradient field with a first orientation and simultaneously to actuate a plurality of further gradient amplifiers of at least one further gradient arrangement, to provide a substantially homogeneous magnetic field, wherein the substantially homogeneous magnetic field is overlaid in the imaging region of the magnet arrangement with the magnetic gradient field.

It is conceivable that the at least one gradient amplifier applies a current flow to exactly one gradient coil of the first gradient arrangement, in order to provide the magnetic gradient field with respect to the first spatial direction. In an exemplary embodiment, the controller is designed to actuate a first gradient amplifier and a second gradient amplifier of the first gradient arrangement to apply asymmetrical current flows to a first gradient coil and a second gradient coil of the first gradient arrangement in order to provide the magnetic gradient field.

In one embodiment, the controller is designed to actuate a plurality of further gradient amplifiers of at least one further gradient arrangement simultaneously to provide a substantially homogeneous magnetic field. This can mean that the current flow through the at least one further gradient arrangement is provided synchronously or simultaneously with the current flow through the first gradient arrangement. The current flow through the at least one further gradient arrangement can intersect, in particular, simultaneously with the current flow through the first gradient arrangement.

In an exemplary embodiment, the controller is designed to actuate a plurality of further gradient amplifiers of a second gradient arrangement and a third gradient arrangement simultaneously in order to provide the substantially homogeneous magnetic field.

By way of the actuation of the plurality of gradient amplifiers of the second gradient arrangement and the third gradient arrangement, a magnetic field strength of the substantially homogeneous magnetic field can advantageously be increased. Thereby, a quality of captured magnetic resonance data can advantageously be improved.

FIG. 1 shows, schematically, an example magnetic resonance apparatus 10. The magnetic resonance apparatus 10 may include a magnet unit 11 which has, for example, a permanent magnet, an electromagnet or a superconducting main magnet 12 for generating a static and homogeneous main magnetic field 13 (BO-magnetic field). In addition, the magnetic resonance apparatus 10 comprises a patient receiving region 14 for accommodating a patient 15. In the present exemplary embodiment, the patient receiving region 14 is designed cylindrical and is surrounded in a circumferential direction by the magnet unit 11. The patient receiving region 14 substantially coincides with an imaging region of the magnetic resonance apparatus 10.

The patient 15 can be positioned by means of a patient positioning apparatus 16 of the magnetic resonance apparatus 10 in the patient receiving region 14. The patient positioning apparatus 16 has a patient table 17, which may be designed to be movable within the patient receiving region 14. The magnet unit 11 also has a gradient coil unit 18 which is used for generating magnetic gradient fields which are used for a position encoding during a magnetic resonance scan. The gradient coil unit 18 is actuated by means of a gradient controller 19 of the magnetic resonance apparatus 10. The gradient controller 19 can have a gradient amplifier 29 (not shown) which is configured to provide a current flow in the gradient coil 18. The magnet unit 11 can further comprise a high frequency antenna unit which is designed in the present exemplary embodiment as a body coil 20 which is permanently integrated into the magnetic resonance apparatus 10. The body coil 20 is configured for exciting atomic nuclei that are situated in the main magnetic field 13 generated by the main magnet 12. The body coil 20 is actuated by a high frequency unit 21 of the magnetic resonance apparatus 10 and radiates high frequency signals into an examination space which is substantially formed by a patient receiving region 14 of the magnetic resonance apparatus 10. The body coil 20 can further also be designed for receiving magnetic resonance signals.

The magnetic resonance apparatus 10 may include a controller 22. The controller 22 may be configured to control the magnetic resonance apparatus 10. For example, the controller 22 may control the main magnet 12, including controlling the gradient controller 19 and the high frequency unit 21. The controller 22 may be configured to control an execution of a sequence, for example, an imaging gradient echo sequence, a TSE sequence or a UTE sequence. In addition, the controller 22 comprises an evaluation unit (evaluator) 28 configured to evaluate digitized magnetic resonance signals which are captured during a magnetic resonance scan. In an exemplary embodiment, the controller 22 includes processing circuitry that is configured to perform one or more functions and/or operations of the controller 22. The controller 22 may include a memory and/or the controller 22 may be configured to access an external memory.

Furthermore, the magnetic resonance apparatus 10 may include a user interface 23 which has a signal connection to the controller 22. Control information such as, for example, imaging parameters and reconstructed magnetic resonance images can be output from the controller 22 via and output interface 24. In an exemplary embodiment, the output interface 24 is a display 24 and the imaging parameters and reconstructed magnetic resonance images may be displayed for a user on the display 24, for example, on at least one monitor, of the user interface 23. The output interface 24 may be a display, speaker, projector, printer, or other output interface. In addition, the user interface 23 has an input interface 25 in which parameters of a magnetic resonance imaging system can be input by the user. The input interface 25 may be a keyboard, mouse, touchscreen display, microphone, or other input interface.

Furthermore, the magnetic resonance apparatus 10 has a local coil 26 which, in the present case, is positioned on a head of the patient 15 and transfers magnetic resonance signals from a volume of a head region to the magnetic resonance apparatus 10. In an exemplary embodiment, the local coil 26 has an electrical connection lead 27 which provides a signal connection to the high frequency unit 21 and the controller 22. However, the local coil 26 can also be connected by a wireless signal connection to the magnetic resonance apparatus 10. Similar to the body coil 20, the local coil 26 can also be designed for an excitation of atomic nuclei and for receiving magnetic resonance signals. For the emitting of high frequency signals, a transmitter of the local coil 26 is actuated by the high frequency unit 21. The local coil 26 can enclose the head of the patient 15 externally circumferentially along a longitudinal axis of the patient 15. The transmitter and/or a receiver of the local coil 26 can be carried, in particular, by a holding element 33 which is able to be positioned relative to a base element of the local coil 26.

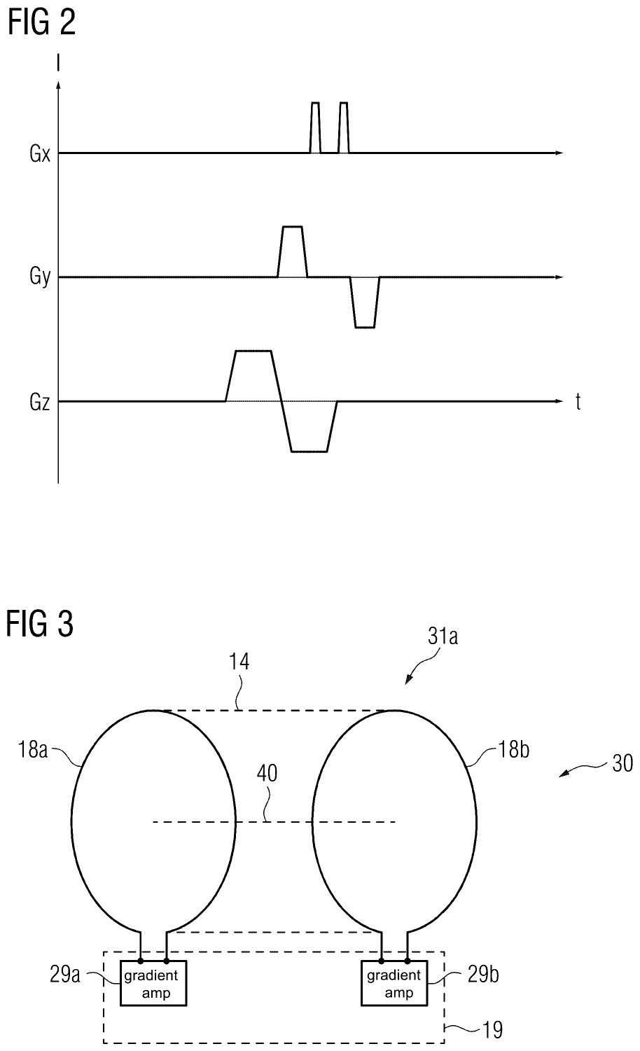

The magnetic resonance apparatuses 10 may have three gradient coils 18 with each of which exactly one gradient amplifier is associated. In order to generate magnetic gradient fields in the X, Y and Z-directions during a magnetic resonance examination, the gradient coils 18 are excited by means of the gradient controller in order to provide the magnetic gradient fields Gx, Gy and Gz. FIG. 2 shows a typical current flow through the gradient coils 18. The magnetic gradient fields in the X, Y and Z-direction are therein overlaid on the static and homogeneous magnetic field of the main magnet 12.

FIG. 3 shows an embodiment of a magnet arrangement 30 according to the disclosure with two gradient coils 18a and 18b (18a-b). In an exemplary embodiment, the magnet arrangement 30 may replace the three gradient coils 18. The gradient coils 18a-b are substantially circular and are arranged planar in parallel-oriented planes which are separated from one another, so that a projection of a first area enclosed by the first gradient coil 18a along a normal vector of the first area and a second area enclosed by the second gradient coil 18b have a non-empty intersection.

In an exemplary embodiment, the gradient coils 18a-b are arranged on opposite ends of a substantially cylindrically designed patient receiving region 14. The gradient coils 18a-b can at least partially enclose the cylindrical patient receiving region 14 along a peripheral direction.

The gradient coil 18a is electrically connected to the gradient amplifier 29a. The gradient amplifier 29a is designed to generate a current flow in the gradient coil 18a. It is conceivable that, for this purpose, the gradient amplifier 29a has a signal connection to the controller 22 (not shown) which is designed to actuate the gradient amplifier 29a accordingly by means of the signal connection. The gradient coil 18b is likewise electrically connected to the gradient amplifier 29b. The gradient amplifier 29b can also have a signal connection to the controller 22 (not shown). The gradient amplifier 29a and the gradient amplifier 29b and also other gradient amplifiers 29 can also be part of a gradient actuating unit (gradient controller) 19 or can be present separately from one another.

The gradient coils 18a-b can, in particular, be designed as a Helmholtz pair. For the generation of a homogeneous magnetic field, the gradient coils 18a-b can be fed with current symmetrically or in the same sense by means of the gradient amplifiers 29a and 29b. In order to provide a magnetic gradient field along the cylindrical axis of the patient receiving region 14 (e.g. the Z-direction), the current flow through the gradient coil 18a can differ from the current flow through the gradient coil 18b. In particular, the current flows through the two gradient coils 18a-b can each have portions or gradient terms which differ during the provision of a magnetic gradient field. For example, the gradient term of the gradient coil 18a can have a negative sign, whereas the gradient term of the gradient coil 18b has a positive sign. In this case, a difference between the current flows through the gradient coil 18a and the second gradient coil 18b can be characterized by the sum of the amounts of the gradient terms of the gradient coils 18a-b. By contrast thereto, the difference between the current flows through the gradient coils 18a-b during provision of a homogeneous magnetic field can substantially correspond to the value zero.

In an exemplary embodiment, the gradient arrangement 31a shown in FIG. 3 may include a third gradient coil 18c (not shown) which is arranged between (e.g. centrally between) the gradient coils 18a-b along the cylinder axis 40. In this case, the gradient arrangement 31a can be designed, in particular, as a Maxwell coil arrangement.

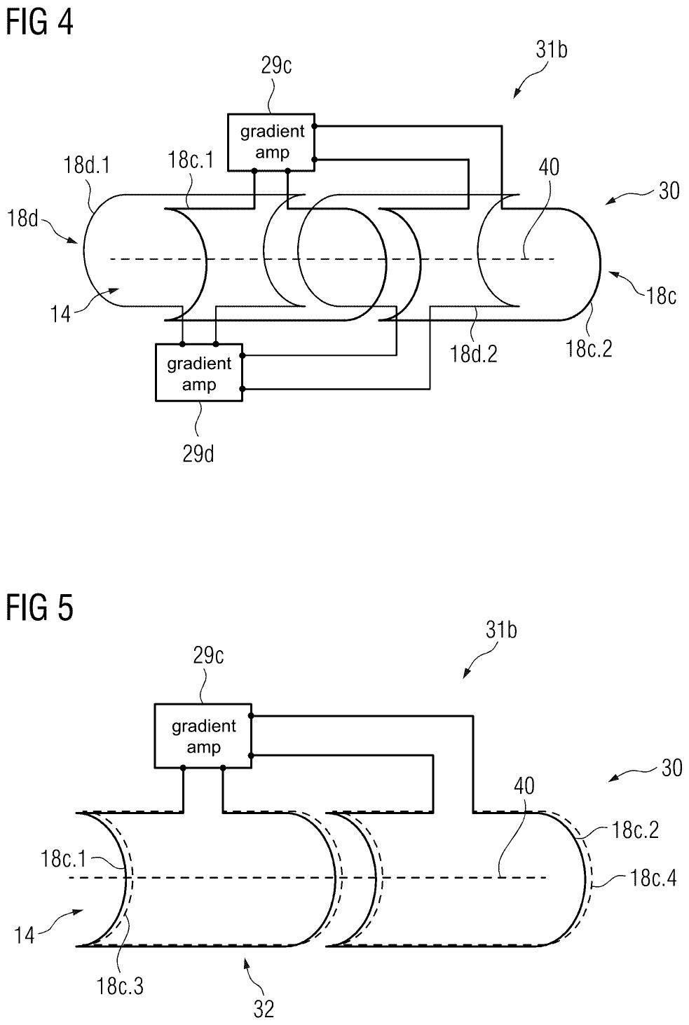

FIG. 4 shows a further embodiment of the magnet arrangement 30 according to the disclosure. In the example shown, the magnet arrangement 30 has a gradient arrangement 31b with the gradient coils 18c and 18d (18c-d) which in the present case are designed as saddle coils. The gradient coils 18c and 18d are electrically unconnected and are positioned opposing one another along an axial direction of the hollow cylindrical magnet arrangement 30. The hollow cylindrical magnet arrangement 30 herein at least partially or completely encloses the cylindrical patient receiving region 14 along the cylindrical axis 40. For example, the gradient coils 18a-b can subdivide the hollow cylindrical magnet arrangement 30 into a left half and a right half and/or into a lower half and an upper half. The left half and the right half and/or the lower half and the upper half can therein be designed symmetrical or can have substantially matching dimensions.

In the present case, the gradient coils 18c and 18d are subdivided into two coil portions 18c.1 and 18c.2, and 18d.1 and 18d.2. The coil portions 18c.1 and 18c.2 of the gradient coil 18c are arranged along the cylindrical axis 40 sequentially or one after the other in the cylindrical magnet arrangement 30. Equally, the coil portions 18d.1 and 18d.2 of the gradient coil 18d are arranged along the cylindrical axis 40 one after the other in the cylindrical magnet arrangement 30. In an exemplary embodiment, the coil portions 18c.1 and 18c.2 are arranged along a sectional plane through the cylindrical axis 40 mirror symmetrically to the coil portions 18d.1 and 18d.2. The coil portions 18c.1 and 18c.2 are electrically connected to the gradient amplifier 29c, whereas the coil portions 18d.1 and 18d.2 are electrically connected to the gradient amplifier 29d.

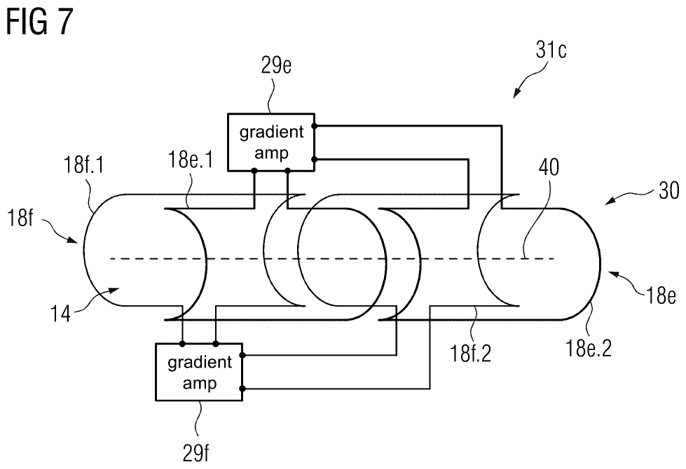

As shown in FIG. 7, the magnet arrangement 30 according to the disclosure can comprise a further gradient arrangement 31c. The gradient arrangement 31c can have gradient coils 18e and 18f which can be subdivided, as shown in FIG. 4, into coil portions 18e.1 and 18e.2, as well as 18f.1 and 18f.2. In an exemplary embodiment, the gradient coils 18e and 18f are designed as saddle coils and are positioned opposing one another in the axial direction of the hollow cylindrical magnet arrangement 30. Similarly, to the gradient arrangement 31b, the gradient coils 18e and 18f can also be electrically connected to dedicated gradient amplifiers 29e and 29f. In an exemplary embodiment, the gradient arrangement 31c is arranged in the magnet arrangement 30 rotated by 90° relative to the gradient arrangement 31b.

As an alternative to the embodiment shown in FIG. 4, the gradient coils may be designed as segment coils in one or more embodiments.

FIG. 5 shows an embodiment of the magnet arrangement 30 according to the disclosure wherein the gradient coil 18c of the gradient arrangement 31b has a secondary coil 32 with the coil portions 18c.3 and 18c.4. The coil portions 18c.3, 18c.4 of the secondary coil 32 are arranged substantially parallel to the coil portions 18c.1 and 18c.2 of the gradient coil 18c and border it along a direction facing away from the patient receiving region 14. The secondary coil 32 is designed to provide a magnetic field in the patient receiving region 14. The secondary coil 32 therefore differs from an electromagnetic screening and/or a screening layer, as is used in conventional magnetic resonance apparatuses (see FIG. 1).

In an exemplary embodiment, the coil portions 18c.3 and 18c.4 of the secondary coil 32 and the coil portions 18c.1 and 18c.2 of the gradient coil 18c are jointly electrically connected to the gradient amplifier 29c. It is also conceivable that the coil portions 18c.3 and 18c.4 of the secondary coil 32 and the coil portions 18c.1 and 18c.2 of the gradient coil 18c are electrically connected to separate gradient amplifiers. Furthermore, each coil portion 18c.1 to 18c.4 can be electrically connected to a separate or dedicated gradient amplifier 29.

In an alternative embodiment, the gradient coil 18c is present as a single piece or undivided, i.e. not in individual coil portions 18c.1 and 18c.2. Equally, the secondary coil can be present as a single piece. It is also conceivable that the gradient coil 18c and the secondary coil 32 are each subdivided into more than two coil portions.

It is conceivable that each gradient coil 18 according to an embodiment described above has a secondary coil 32. In particular, the gradient coil 18d, and also the gradient coils 18e and/or 18f of the gradient arrangement 31c (not shown) can have secondary coils 32. It is also conceivable that the gradient coils 18a and 18b (see FIG. 3) also have secondary coils 32 each of which is arranged substantially parallel to the gradient coils 18a and 18b and border them in a direction facing away from the patient receiving region 14.

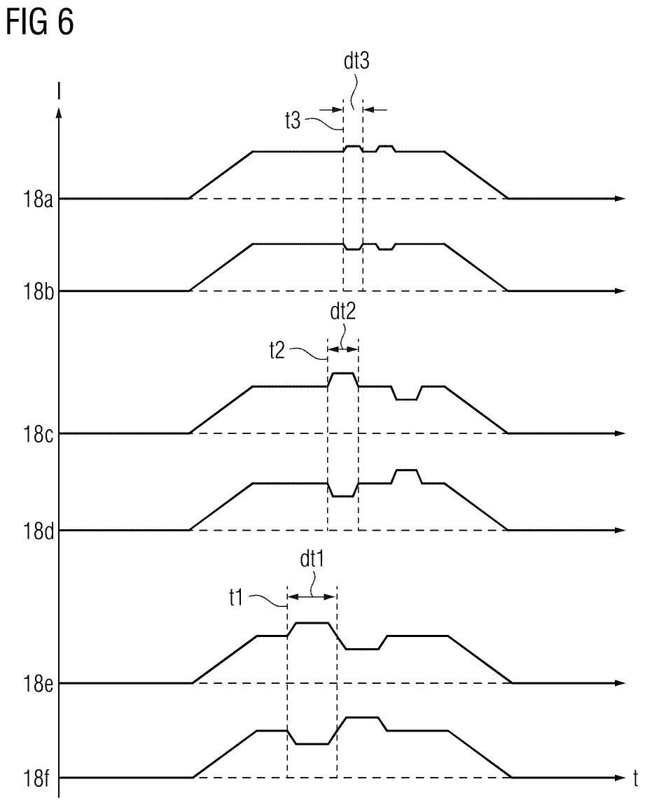

FIG. 6 shows schematically how the gradient arrangements 31a, 31b and 31c (31a-c) of the magnet arrangement 30 according to the disclosure are able to be actuated by means of a plurality of gradient amplifiers in order to provide, in an alternating manner, a homogenous magnetic field and a magnetic gradient field or a homogenous magnetic field with overlaid magnetic gradient fields.

In an exemplary embodiment, the gradient arrangements 31a-c each have two gradient coils 18 and two gradient amplifiers 29. Each gradient amplifier 29a-f (not shown) is electrically connected to a gradient coil 18a-f according to an embodiment described above.

In an exemplary embodiment, a homogeneous magnetic field is provided wherein the magnetic fields overlap the gradient arrangements 31a-c. It is conceivable that each gradient arrangement 31a, 31b and 31c provides a substantially homogeneous magnetic field, said fields overlapping in the patient receiving region 14. A magnetic field strength of the homogeneous magnetic field can result by addition of the magnetic field strengths of the magnetic fields of the gradient arrangements 31a-c. For the provision of the homogeneous magnetic fields, two gradient coils 18a and 18b, 18c and 18d, and 18e and 18f of the gradient arrangements 31a-c can have a current flow I applied, in each case, by means of the gradient amplifiers 29a and 29b, and 29c and 29d, as well as 29e and 29f, symmetrically or with the same sense. For example, the gradient coils 18a and 18b can have a same-sense current flow I applied to them by means of the gradient amplifier 29a and 29b. Equally, the gradient coils 18c and 18d can be supplied with a symmetrical current flow I by means of the gradient amplifier 29c and 29d.

In the example shown in FIG. 6, the gradient coils 18a and 18b generate a homogeneous magnetic field in the patient receiving region 14 when the gradient amplifiers 29a and 29b provide a symmetrical current flow I in the gradient coils 18a and 18b. The gradient coils 18a and 18b can herein act, in particular, as Helmholtz coils. The current flow I through the gradient coils 18a and 18b can have flanks which can result from a ramp-up and ramp-down behavior of the gradient amplifier 29a and 29b. The time-dependent current flow I can therefore have an approximately trapezoid form.

In order to generate the homogeneous magnetic field, symmetrical current flows I can be provided, at the same time, in the gradient coils 18c and 18d, and 18e and 18f. The homogeneous magnetic fields generated by the gradient coils 18a-f become overlaid in the patient receiving region 14.

It is also conceivable that the magnetic fields provided by the gradient arrangements 31a, 31b and 31c have gradients (magnetic gradient fields) which become overlaid to form overall a homogeneous magnetic field in the patient receiving region 14.

It is further conceivable that only one gradient arrangement 31 or two gradient arrangements provide a homogeneous magnetic field in the patient receiving region 14. In this case, the gradient coils 18 of the one gradient arrangement 31 or both gradient arrangements 31 which do not provide a homogeneous magnetic field can have a current flow I applied asymmetrically or have no current flow I. For example, the gradient coils 18a-b and 18c-d each have symmetrical current flows I applied by means of the gradient amplifiers 29a-b and 29c-d in order to provide a homogeneous magnetic field in the patient receiving region 14. By contrast therewith, the gradient coils 18e-f have asymmetrical current flows I applied by means of the gradient amplifiers 29e-f in order to provide a magnetic gradient field with an orientation in the X-direction in the patient receiving region 14.

In FIG. 6, an exemplary embodiment of the magnet arrangement 30 according to the disclosure is shown wherein the gradient arrangements 31a-c have a current flow I applied jointly at selected time points in order to provide a homogeneous magnetic field in the patient receiving region 14. For the spatial encoding of captured magnetic resonance signals during an imaging sequence, however, short-lived magnetic gradient fields are typically needed along the three spatial directions of a Cartesian coordinate system. Therefore, the gradient amplifiers 29a-f of the gradient arrangements 31a-c are designed, in the present case, to invert a small portion of the current flow I (gradient term) in opposingly positioned gradient coils 18 of a gradient arrangement 31 briefly in order to generate a magnetic gradient field in a desired spatial direction.

As FIG. 6 shows by way of example, the current flow I through the gradient coil 18c can be raised briefly by means of the gradient amplifier 29c at a time point t2, whereas the current flow I through the gradient coil 18d is lowered briefly by means of the gradient amplifier 29d at the time point t2. Thereby, a magnetic gradient field with an orientation in the X-direction can be provided without causing a significant change to the magnetic field strength of the underlying homogeneous magnetic field. Similarly, a current flow through the gradient coil 18e can be raised briefly by means of the gradient amplifier 29e at a time point t1, whereas the current flow I through the gradient coil 18f is lowered briefly by means of the gradient amplifier 29f at the time point t1 in order to provide a magnetic gradient field with an orientation in the Y-direction.

In a similar way, a magnetic gradient field in the Z-direction can be provided by means of the magnet arrangement 30 according to the disclosure. A time point t1 at which a portion of the current flow I through opposingly positioned gradient coils 18 of a gradient arrangement 31 is briefly inverted can therein be specified dependent upon a magnetic resonance examination or imaging sequence that is to be carried out. The time points t1, t2, t3 can therein differ. It is also conceivable that two or three of the time points t1, t2 and t3 coincide.

The timespans dt1, dt2 and dt3 can characterize timespans in which the current flows I are varied by means of the gradient amplifiers in order to provide a magnetic gradient field. It is conceivable that the timespans dt1, dt2 and dt3 at least partially overlap one another temporally. The timespans dt1, dt2 and dt3 can however also follow one another temporally or be mutually spaced temporally. In one embodiment, the durations of the timespans dt1, dt2 and dt3 mutually differ.

Naturally, the current flows I through the gradient coils 18a-f represented in FIG. 6 are to be understood as purely exemplary. The current flows I and the magnetic fields generated can vary dependent upon the imaging sequence, the magnetic resonance examination and also upon an object being examined and/or a specific geometry of the magnetic resonance apparatus.

It is conceivable, in particular, that the geometry of the magnetic resonance apparatus deviates from a conventional cylindrical shape. For example, the magnetic resonance apparatus can be configured as a C-type or an “open” scanner. The gradient coils can accordingly have a substantially planar form. In an exemplary embodiment, the gradient coils are arranged, in this case, on two substantially opposingly positioned sides of the imaging volume or the patient receiving region 14 and have adapted winding patterns in order to generate magnetic gradient fields in desired spatial directions. For example, a first gradient coil with a first gradient amplifier of a first gradient arrangement and a second gradient coil with a second gradient amplifier of the first gradient arrangement are arranged on opposite sides of an imaging volume and/or the patient receiving region 14 of the magnet arrangement and spaced apart from one another.

The magnet arrangement 30 according to the disclosure can naturally comprise further components that magnetic resonance apparatuses 10 typically have. It is also conceivable that, in place of the cylindrical construction, the magnetic resonance apparatus 10 has a C-shaped, triangular or asymmetrical construction of the components generating the magnetic field. The magnetic resonance apparatus 10 can be, in particular, a dedicated magnetic resonance apparatus 10 which is designed to carry out a magnetic resonance imaging of the jaw region of a standing or sitting patient 15.

Although the disclosure has been illustrated and described in detail with the preferred exemplary embodiments, the disclosure is nevertheless not restricted by the examples given and other variations can be derived therefrom by a person skilled in the art without departing from the protective scope of the disclosure.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure.

Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A magnet arrangement for a magnetic resonance apparatus operable to capture magnetic resonance data from an object, the magnet arrangement comprising:
a plurality of gradient coils; and
a plurality of gradient amplifiers configured to variably set a current flow in the plurality of gradient coils, wherein each gradient coil of the at least one gradient arrangement is electrically connected to a gradient amplifier of the plurality of gradient amplifiers, the plurality of gradient coils and the plurality of gradient amplifiers forming a gradient arrangement,
wherein the magnet arrangement is configured to provide, by the plurality of gradient amplifiers, in an alternating manner, a substantially homogeneous magnetic field and a magnetic gradient field.

2. The magnet arrangement as claimed in claim 1, wherein the gradient arrangement comprises a first gradient coil and a second gradient coil, the first gradient coil and the second gradient coil being substantially circular and arranged planar in parallel-oriented planes separated from one another, such that a projection of a first area enclosed by the first gradient coil along a normal vector of the first area and a second area enclosed by the second gradient coil have a non-empty intersection, wherein the first gradient coil is electrically connected to a first gradient amplifier of the plurality of gradient amplifiers and the second gradient coil is electrically connected to a second gradient amplifier of the plurality of gradient amplifiers.

3. The magnet arrangement as claimed in claim 1, further comprising a second gradient arrangement with a plurality of second gradient coils and a plurality of second gradient amplifiers, wherein:
at least two gradient coils of the plurality of second gradient coils are each electrically connected to one gradient amplifier of the plurality of second gradient amplifiers, and
the magnet arrangement is configured to provide, by the plurality of second gradient amplifiers, in an alternating manner, a substantially homogeneous magnetic field and a second magnetic gradient field, an orientation of the second magnetic gradient field being different from an orientation of the magnetic gradient field.

4. The magnet arrangement as claimed in claim 3, further comprising a third gradient arrangement with a plurality of third gradient coils and a plurality of third gradient amplifiers, wherein:
at least two gradient coils of the plurality of third gradient coils are each electrically connected to one gradient amplifier of the plurality of third gradient amplifiers, and
the magnet arrangement is configured to provide, by the plurality of third gradient amplifiers, in an alternating manner, a substantially homogeneous magnetic field and a third magnetic gradient field, an orientation of the third magnetic gradient field being from an orientation of the magnetic gradient field and/or of the second magnetic gradient field.

5. The magnet arrangement as claimed in claim 4, wherein the plurality of second gradient coils and/or the plurality of third gradient coils are configured as saddle coils.

6. The magnet arrangement as claimed in claim 4, wherein the plurality of second gradient coils and/or the plurality of third gradient coils are configured as segment coils.

7. The magnet arrangement as claimed in claim 3, wherein the magnet arrangement has a hollow cylindrical form, at least one gradient coil of the second gradient arrangement including a first coil portion and a second coil portion, wherein the first coil portion and the second coil portion are arranged disjointed from one another along an axial direction of the hollow cylindrical magnet arrangement, following one another, and wherein the first coil portion and the second coil portion are jointly electrically connected to exactly one gradient amplifier of the second gradient arrangement.

8. The magnet arrangement as claimed in claim 1, wherein at least one gradient coil of the gradient arrangement has a secondary coil arranged parallel to the gradient coil and borders the at least one gradient coil in a direction facing away from an imaging region of the magnet arrangement, wherein the secondary coil and the at least one gradient coil are jointly electrically connected to a gradient amplifier of the plurality of gradient amplifiers.

9. The magnet arrangement as claimed in claim 4, wherein the gradient arrangement, the second gradient arrangement and/or the third gradient arrangement are electromagnetically unscreened.

10. The magnet arrangement as claimed in claim 4, wherein the gradient arrangement, the second gradient arrangement and/or the third gradient arrangement replace a main magnet for generating a static homogeneous magnetic field.

11. A magnetic resonance apparatus operable to capture magnetic resonance data from an object, comprising:
a magnet arrangement including
a plurality of gradient coils; and
a plurality of gradient amplifiers configured to variably set a current flow in the plurality of gradient coils, wherein each gradient coil of the at least one gradient arrangement is electrically connected to a gradient amplifier of the plurality of gradient amplifiers, the plurality of gradient coils and the plurality of gradient amplimers forming a gradient arrangement, wherein the magnet arrangement is configured to provide, by the plurality of gradient amplifiers, in an alternating manner, a substantially homogeneous magnetic field and a magnetic gradient field; and
a controller configured to actuate the plurality of gradient amplifiers to variably set a current flow in the plurality of gradient coils to provide, in an alternating manner, a substantially homogeneous magnetic field and a magnetic gradient field in an imaging region of the magnet arrangement.

12. The magnetic resonance apparatus as claimed in claim 11, wherein the controller is configured to convert symmetrical current flows through the plurality of gradient coils of the gradient arrangement using the plurality of gradient amplifiers into asymmetrical current flows to provide a changeover from a homogeneous magnetic field to a magnetic gradient field.

13. The magnetic resonance apparatus as claimed in claim 11, further comprising a second gradient arrangement with a plurality of second gradient coils and a plurality of second gradient amplifiers, wherein the controller is configured to simultaneously actuate:
at least one gradient amplifier of the gradient arrangement to set a current flow through the gradient arrangement to provide a magnetic gradient field with a first orientation, and
the plurality of second gradient amplifiers to provide a homogeneous magnetic field overlaid in the imaging region of the magnet arrangement with the magnetic gradient field.

14. The magnetic resonance apparatus as claimed in claim 13, further comprising a third gradient arrangement with a plurality of third gradient coils and a plurality of third gradient amplifiers, wherein the controller is configured to simultaneously actuate the plurality of second gradient amplifiers and the plurality of third gradient amplifiers to provide the homogeneous magnetic field.
